SEQUENCE LISTING (2 sequences)

SEQ ID NO: 1 - DNA, 23 bp, Artificial Sequence
  Description of Artificial Sequence  STAT-3 specific oligonucleotide
gatccttctggaattcctagatc

SEQ ID NO: 2 - DNA, 24 bp, Artificial Sequence
  Description of Artificial Sequence Mutant STAT-3 specific oligonucleotide
gatccttctgggccgtcctagatc

(19) United States
(12) Patent Application Publication
(10) Pub. No.: US 2004/0203067 A1
(43) Pub. Date: Oct. 14, 2004

(54) Methods for cancer prognosis and screening antiproliferative agents

(75) Inventors: E. Premkumar Reddy, Villanova, PA (US); Priya Chaturvedi, King of Prussia, PA (US); M.V. Ramana Reddy, Upper Darby, PA (US); John R. Jenkins, Kent (GB)

Correspondence Address: DRINKER BIDDLE & REATH, ONE LOGAN SQUARE, 18TH AND CHERRY STREETS, PHILADELPHIA, PA, 19103-6996, US

(73) Assignee: Temple University - Of The Commonwealth System of Higher Education, Philadelphia, PA

(21) Appl. No.: 10/846,369

(22) Filed: May 13, 2004

Related U.S. Application Data
(63) Continuation of application No. 09/670,128, filed on Sep. 26, 2000.
Application No. 09/670,128 is a continuation of application No. PCT/US99/06514, filed on Mar. 25, 1999.
(60) Provisional application No. 60/079,755, filed on Mar. 27, 1998.

(51) Int. Cl. C12Q 1/68

(52) U.S. Cl. 435/6

(57) ABSTRACT

The invention provides prognostic methods which comprise determining the level of expression of the gene c-fyn, or the level of activated STAT-3 protein. Because the relative level of c-fyn expression and level of activated STAT-3 protein correlate with the presence of malignant potential and therefore patient prognosis, these markers may be used to make treatment decisions, to predict patient outcome, and to predict the risk of cancer in disease-free individuals. The invention further provides a method for identifying anticancer drugs in which inhibitors of cell proliferation are identified as inhibitors of Src-mediated STAT phosphorylation.

### CROSS-REFERENCE TO RELATED APPLICATION

This application is a continuation of co-pending U.S. application Ser. No. 09/670,128, filed Sep. 26, 2000, which is a continuation of International Application No. PCT/US99/06514 filed Mar. 25, 1999, which claims priority from U.S. Provisional Application No. 60/079,755, filed Mar. 27, 1998, now abandoned.

### REFERENCE TO GOVERNMENT GRANT

The invention described herein was supported in part by National Institutes of Health grant CA68239. The U.S. government has certain rights in the invention.

### FIELD OF THE INVENTION

The invention relates to methods for the prognosis of cancer, and for the design of anticancer agents.

### BACKGROUND OF THE INVENTION

Signal Transducers and Activators of Transcription (STATs)

Signal transducers and activators of transcription (STATs) are a family of latent cytoplasmic proteins that, when activated, participate in gene control upon stimulus from various extracellular proteins. Hematopoietic cell growth is mediated by a group of soluble growth factors, which bind to their cognate receptors and trigger the activation of STATs (Ihle and Kerr, Trends Genetics 11:69-74 (1995)). STATs were originally described by Darnell and co-workers (Darnell et al., Science 264:1415-1421 (1994); Darnell, Rec. Prog. Horm. Res. 51:391-403 (1996); Darnell, Science 277:1630-1635 (1997)) as ligand-induced transcription factors in interferon-treated cells. Subsequent studies by a number of groups showed that STATs play a critical role in signal transduction pathways associated with several cytokines and neurokines including the interleukins, the interferons, erythropoietin, prolactin, growth hormone, oncostatin M (OSM), and ciliary neurotrophic factor (CNTF) (Eilers et al., Mol. Cell. Biol. 14:1364-1373 (1994); Lehmann et al., J. Immunol. 153:165-172 (1994); Silva et al., Endocrinology 10:508-518 (1996)).

To date, seven mammalian genes that code for different STATs have been identified, all of which encode for proteins of 750-850 amino acids long and are characterized by the presence of a DNA-binding domain followed by putative SH3 and SH2 domains (Darnell, Rec. Prog. Horm. Res. 51:391-403 (1996)). These proteins, which are normally localized in the cytoplasm, are activated when phosphorylated on a single tyrosine located around reside 700, which facilitates their dimerization and translocation to the nucleus (Darnell et al., Science 264:1415-1421 (1994); Schindler and Darnell, Ann. Rev. Biochem. 64:621-651 (1995)).

A large number of cytokines, neurokines and interferons have been recently shown to interact with their receptors and trigger the activation of proliferative and differentiation pathways in cells (Kishimoto et al., Cell 76:253-262 (1994); Ihle, Adv. Immunol. 60:1-35 (1995); Sachs and Lotem, Proc. Soc. Exper. Biol. Med. 206:170-175 (1994)). Current models suggest that interaction of a cytokine with its receptor induces receptor dimerization which increases the affinity of the cytoplasmic domain of the receptor for Janus kinases (JAKs) (Leaman et al., FASB J. 10:1578-1588 (1996); Ihle, Adv. Immunol. 60:1-35 (1995)). This results in a ligand-dependent increase of a complex that contains the receptors and JAK kinases which have been activated through an event associated with tyrosine phosphorylation. The activated JAK kinases appear to subsequently phosphorylate the C-terminal end of receptors that serve as the docking sites for STATs. The receptor bound STAT is then phosphorylated on tyrosine, which in turn leads to its activation (Schindler and Darnell, Ann. Rev. Biochem. 64:621-651 (1995); Leaman et al., FASEB J. 10:1578-1588 (1996); Ihle and Kerr, Trends Genetics 11:69-74 (1995); Kohlhuber et al., Mol. Cell. Biol. 17:695-706 (1997)).

However, accumulating evidence suggests that STAT activation may not be mediated by JAK kinases. For example, the activated JAK kinases do not seem to exhibit specificity for a particular STAT as different receptors activate a common STAT, even though they activate distinctively different JAK kinases (Kotenko et al., J. Biol. Chem. 271:17174-17182 (1996); Kohlhuber et al., 1997). Thus, the specificity for STAT phosphorylation appears to be determined by the docking sites for STATs that are present in the receptor molecules and not JAK kinases. This leaves the question open as to which tyrosine kinases mediate the phosphorylation of STATs.

Cancer Prognosis

The ability to identify cancer patients with more aggressive diseases is crucial to planning adequate treatment. Selecting an appropriate course of therapy requires an accurate determination of the cancer's malignant potential. With this purpose in mind, several pathologic tumor features have been considered so far, including histologic type, grade of differentiation, depth of invasion, and extent of lymph nodal metastases. Unfortunately, these factors do not always allow a sufficiently accurate stratification of cancer patients. Such parameters also have questionable reproducibility.

The histological grading of tumors in particular is fraught with uncertainties. Grading is typically carried out by examination of the character and appearance of tumor sections by skilled pathologists. A significant problem in the use of histologic criteria when determining the prognosis and types of treatment for cancer is the degree of interobserver and intraobserver variability in reading the same specimens. Determinations are necessarily subjective. In addition, there may be heterogeneity within the tumor itself in both primary and metastatic sites. It may become necessary to obtain the opinion of several pathologists to reach a consensus on individual tumor grade.

There is great need for a simple laboratory test which is a consistent predictor of clinical outcome in cancer. An early and accurate prognostic assessment allows the correct therapeutic intervention at the earliest stages of the disease, thereby maximizing the prospects for a positive therapeutic outcome. What is needed is a prognostic method which can, at an early disease stage, identify the aggressiveness of an individual patient's disease, before initiation of therapy. This will permit maximum flexibility in selecting the appropriate course of therapy.

Anticancer Drug Discovery

Although many classes of antineoplastic drugs have been developed for use in clinical practice, cancer is still a major cause of mortality and morbidity. There is, therefore, a great need to identify additional compounds which are potentially useful for the treatment of cancers. The most important step in an anticancer drug discovery program is mass screening, in which large numbers of test compounds (for example, combinatorial libraries) are tested for a relevant biological activity. The mass screening is used to identify a manageable number of drug candidates for animal, and potentially clinical testing. An effective screening assay must have a high throughput capacity and must measure a biological activity which is predictive of antitumor activity.

### SUMMARY OF THE INVENTION

According to one embodiment of the invention, a method for determining a prognosis in a patient afflicted with cancer is provided, comprising determining the expression level of the c-fyn gene in a sample from the patient. An increased level of c-fyn expression is indicative of an unfavorable prognosis.

According to another embodiment of the invention, a method for tumor grading is provided, comprising determining the expression level of the c-fyn gene in a sample from the patient. The level of c-fyn expression is indicative of the grade of the cancer.

According to another embodiment of the invention, a method for determining the metastatic potential of a cancer in an afflicted patient is provided, comprising determining the level of c-fyn expression in a sample from the patient. An increased level of c-fyn expression is indicative of the metastatic potential of the tumor.

According to another embodiment of the invention, a method for determining a prognosis in a patient afflicted with cancer is provided, comprising determining the level of activated STAT-3 protein in a sample from the patient, an increased level of said protein being indicative of an unfavorable prognosis.

According to another embodiment of the invention, a method for tumor grading is provided, comprising determining the level of activated STAT-3 protein in a sample from the patient. The level of activated STAT-3 protein is indicative of the grade of the cancer.

According to another embodiment of the invention, a method for determining the metastatic potential of a cancer in an afflicted patient is provided, comprising determining the level of activated STAT-3 protein in a sample from the patient. An increased level of activated STAT-3 protein is indicative of the metastatic potential of the tumor.

The invention also provides a method for identifying compounds that inhibit cell proliferation comprising measuring the ability of a test compound to inhibit Src kinase-mediated STAT phosphorylation, wherein inhibitors of cell proliferation are identified as inhibitors of Src-mediated STAT phosphorylation.

The Src kinase can be any member of the Src family. In a preferred embodiment the Src kinase is c-Src, c-Fyn, or c-Fgr; in a most preferred embodiment the Src kinase is c-Src. In some embodiments, the STAT is STAT-3 or STAT-5; in a preferred embodiment, the STAT is STAT-3.

The method according to the invention may be practiced using a cell free system, mammalian cells, or yeast cells; in a preferred embodiment, the Src-mediated STAT phosphorylation is measured in recombinant fission yeast cells.

In some embodiments, the inhibition of STAT phosphorylation is measured as the level of expression of a reporter gene under the control of a STAT dependent promoter element; in a preferred embodiment the reporter gene is green fluorescent protein (GFP).

In some embodiments, the inhibition of STAT phosphorylation is measured directly; in some embodiments the level of STAT phosphorylation is measured in a DNA binding assay.

### DESCRIPTION OF THE FIGURES

FIG. 1 shows Western blots of lysates from 32Dcl3 cells. Total cell lysates were probed with anti-Src monoclonal antibody (FIG. 1A) or anti-STAT-3 antibody (FIG. 1B). Lysates were immunoprecipitated with Anti-Src monoclonal antibody or with preimmune serum (PI) and probed with anti-STAT-3 antibody (FIG. 1C). Lysates were immunoprecipitated with anti-Src (N-16) antibody or with antibody preincubated with excess Src peptide and probed with anti-STAT-3 antibody (FIG. 1D).

FIG. 2 shows Western blots of cell lysates from 32Dcl3 cells, 32D/v-Src cells, and 32D/AMSrc cells. Total cell lysates were probed with anti-FLAG (FLAG peptide) antibodies (FIG. 2A). Lysates were immunoprecipitated with anti-FLAG antibody, anti-Src antibody, or preimmune serum (PI), and probed with anti-STAT-3 antibody (FIG. 2B). Lysates from IL-3 stimulated cells were immunoprecipitated with anti-STAT-3 antibodies and probed with anti-STAT-3 or 4G10 (anti-phosphotyrosine) antibodies (FIG. 2C).

FIG. 3 is a DNA-binding assay showing the DNA binding activity of STAT-3 in 32Dcl3 and 32D/AMSrc cells.

FIG. 4 is a Western blot of lysates from 32Dcl3, 32D/AMSrc, and 32D/vSrc cells which have been immunoprecipitated with anti-JAK2 antibody and probed with anti-JAK-2 antibody (FIG. 4A) or monoclonal antibody 4G10 (FIG. 4B).

FIG. 5 is a growth curve of 32Dcl3 cells and 32D/AMSrc cells in the presence or absence of tetracycline.

FIG. 6 shows Western blots of cell lysates from 32Dcl3, 32D/AMSrc, and 32D/JAK2KE cells probed with anti-FLAG (FLAG tag) antibody (FIG. 6A), anti-STAT-3 antibody (FIG. 6B), or anti-phospho-STAT-3 antibody (FIG. 6C).

FIG. 7 is a Western blot of cell lysates of the breast cancer cell lines BT20, 126, T47D, MCF-7, ZR75, BT474, 415, and 435, stained with antibody to STAT-3 or antibody to phospho-STAT-3, or with anti-estrogen receptor antibody (ER).

FIG. 8 is a Western blot of cell lysates of the breast cancer cell lines BT20, 126, and 435, and the prostate tumor cell lines LNCAP, DU145 and PC3, stained with antibody to STAT-3, antibody to phospho-STAT-3, or antibody to the protein FYN.

FIG. 9 is a Western blot of lysates of BT20 breast cancer cells treated with or without the experimental anti-breast cancer agent FRI-20, stained with antibody to phospho-STAT-3 (top panel) or antibody to STAT-3 (bottom panel).

### DEFINITIONS

“Allele” refers to one or more alternative forms of a gene occupying a given locus on a chromosome.

“Affected tissue” means tissue which, through visual or other examination, is believed to contain a purported cancerous or precancerous lesion.

“Expression”, with respect to a gene, means the realization of genetic information encoded in the gene to produce a functional RNA or protein. The term is thus used in its broadest sense, unless indicated to the contrary, to include either transcription or translation.

“Expression level”, with respect to a gene, means not only an absolute expression level, but also a relative expression level as determined by comparison with a standard level of gene expression.

“Grading”, with respect to a tumor sample, means a classification of the perceived degree of malignancy. In grading tumor samples, a pathologist or other observer evaluates the degree of differentiation (e. g. grade 1, well differentiated, grade 2, moderately differentiated, grade 3, poorly differentiated) of the tissue.

“Gynecologic cancer” means a tumor arising in the uterus, ovary, cervix, vagina, vulva, or fallopian tube, as well as gestational trophoblastic disease.

“c-fyn gene” means the gene which encodes the FYN protein, the cDNA of which is set forth in Rigley et al., J. Immunol. 154(3):1136-1145 (1995), and all allelic variations and mutants thereof.

“STAT-3 gene” means the gene which encodes the STAT-3 protein, the cDNA of which is set forth in Akira et al., Cell 77(1):63-71(1994), and all allelic variations and mutants thereof.

“FYN protein” means the translation product of the c-fyn gene, including all allelic variations and mutants thereof. The FYN amino acid sequence is set forth by Rigley et al.

“STAT-3 protein” means the translation product of the STAT-3 gene, including all allelic variations and mutants thereof. The STAT-3 amino acid sequence is set forth by Akira et al.

“Activated STAT-3” of “phosphorylated STAT-3” or “phospho-STAT-3”, with reference to the STAT-3 protein, means the phosphorylated form of STAT-3 which is active as a transcription factor.

“Prognosis” is used according to its ordinary medical meaning, that is, the prospect of recovery from a disease.

“Src kinase” means a tyrosine kinase from the Src family, including but not limited to c-Src, c-Yes, c-Fgr, Fyn, Lck, Hck, Lyn, and Blk.

### DETAILED DESCRIPTION OF THE INVENTION

We have investigated the nature of tyrosine kinases that mediate STAT phosphorylation during cytokine-mediated activation of cell proliferation. We have demonstrated that interaction of cytokines with their receptors leads to the activation of c-Src kinase activity, which in turn facilitates the binding of c-Src to STAT-3. This association leads to the phosphorylation of STAT-3, allowing this transcription factor to translocate to the nucleus. Thus, STAT-3 is activated by c-Src. We have demonstrated that, contrary to prior reports, JAK and STAT phosphorylation events are mediated by two distinct pathways. The activation of JAKs and STATs now appear to be two independent but related events, which dictate two separate biological outcomes.

Our finding that c-Src mediates cytokine-induced cell proliferation by activating STAT-3 provides a useful target for therapeutic intervention in the treatment of proliferative disorders, particularly cancer.

Assays which measure the specific inhibition of Src-mediated STAT phosphorylation can be used to screen large numbers of compounds for antiproliferative activity. It is particularly advantageous to use the assays according to the invention to screen combinatorial libraries for antiproliferative activity. The compounds identified in these assays can then be tested as promising anticancer therapeutics.

Assays according to the invention can have many formats, and include any method which measures the specific inhibition of STAT protein tyrosine phorylation by a Src family kinase.

In one class of assay, a reporter construct is provided in which expression of a marker gene (such as green fluorescent protein) is under the control of a STAT dependent promoter element. A second construct provides an activating Src family kinase gene, and a third construct provides a STAT gene. The constructs are most advantageously combined in a recombinant cell. In the absence of inhibitory activity, the Src kinase activates the STAT protein which leads to expression of the marker gene. Inhibitory compounds are identified as compounds which decrease the expression of the marker gene.

Another class of assay directly measures the level of Src directed tyrosine phosphorylation of STAT in the presence and absence of test compounds. Phosphorylated STAT can be measured using antibodies specific for the tyrosine phosphorylated form of the target peptide motif. There are a number of variations of this basic approach.

In some variations a modified STAT reporter is created, in which the STAT protein lacks nuclear transport signal sequences but includes a membrane transport signal sequence. The STAT reporter protein is expressed in a cell along with Src kinase, and extracellular levels of secreted phosphorylated STAT reporter protein are monitored. Examples of further variations include:

The STAT reporter can be reduced in size by removing all protein sequences that are not required for recognition and phosphorylation by Src kinases.

Multiple Src phosphorylation sites can be included in a single molecule.

Additional epitopes can be included for simultaneous measurement of tyrosine phosphate and total reporter protein.

Determinants can be included for use as anchors to attach the reporter protein to surfaces—for example to a microtitre well.

Protein determinants can be added to anchor the reporter protein to the yeast cell surface. For example, a single chain antibody (SCA) recognizing an epitope on the yeast cell surface can be generated, and the antigen binding site from this SCA can be incorporated into the STAT reporter. This hybrid protein will be secreted, and the secreted protein will attach to the yeast cell surface, facilitating subsequent assay readout by for example ELISA or FACS analysis.

This approach can also be applied to mammalian cell culture systems.

We have also found, quite unexpectedly, that the level of activated STAT-3 (i.e., phosphorylated STAT-3) correlates with the severity of malignancy in cancers. For example, we have found that phosphorylated STAT-3 is present in cells of tumors which exhibit hormone-independent growth, that is, tumors which are generally more malignant, more metastatic, and more likely to be associated with a significantly poorer patient prognosis. On the other hand, we have found that phosphorylated STAT-3 is barely detectable, or entirely absent, in tumors which exhibit hormone-dependent growth. Tumors which exhibit a hormone-dependent growth pattern are less malignant, less metastatic, and generally more readily treatable than hormone-independent tumors. Upon histological grading, they are typically characterized by scores reflecting a less severe malignancy. For example, the retention of hormone receptors in breast tumor cells suggests a more differentiated state of the neoplasia, better prognosis, and longer survival. See, Brooks et al., “Breast Cancer Biology” in Encyclopedia of Human Biology, vol. 2, Academic Press (1991), p. 59. The oestrogenic steroid hormones are known to play critical roles in breast cancer progression. Oestrogen and progesterone receptors when both detected in breast cancers, are considered good prognostic factors.

We have found that the expression level of c-fyn, a member of the Src kinase family, parallels the level of activated STAT-3. We have found that cells of tumors exhibiting hormone-independent growth are characterized by substantial c-fyn expression. We have found that c-fyn expression is barely detectable, or entirely absent, in tumors which exhibit hormone-dependent growth.

According to the present invention, improved methods are provided for the prognosis of cancers, based on activated STAT-3 levels and/or c-fyn expression levels. Such cancers include, but are not limited to, cancers characterized by solid tumors, such as tumors of the breast, prostate and lung; gynecological cancers such as endometrial and ovarian cancers. Cancers for which prognosis may be determined according to the practice of the invention also include those cancers which are not characterized by the occurrence of solid tumors, such as the various hematologic neoplasms, most notably leukemias and lymphomas. Cells of tissue with the greatest malignant potential will be characterized by the presence of FYN or activated STAT-3 molecules.

The prognostic method of the present invention may be used in the selection of candidates for a less aggressive surgical treatment, without decreasing their chance of cure, as well as being helpful for the identification of high risk patients, to whom every surgical effort should be attempted and post-surgical treatment given.

Thus, the c-fyn expression and/or STAT-3 phosphorylation may serve as convenient molecular markers to replace or augment conventional prognostic techniques. An important advantage of the these methods over classical surgical pathologic parameters as a prognostic factor is that the former can be determined at the time of the initial diagnosis, before any therapy is initiated. For patients not previously treated by radiotherapy or chemotherapy, phospho-STAT-3 and/or c-fyn expression can be used to identify tumors with a tendency to behave aggressively.

An early accurate determination of the aggressiveness of disease in an individual patient is a necessary part of designing a course of treatment. In cases where the test method of the invention identifies a poor prognosis, adjuvant therapy, such as radiation therapy or chemotherapy, may be initiated. This more aggressive treatment should increase the patient's chance of survival. STAT-3 phosphorylation and c-fyn expression level, even potentially in early stages of the disease, is believed to be reflective of the malignant potential of the patient's carcinoma and the aggressiveness of the ensuing disease course. This form of “molecular based” prognosis can be evaluated more consistently than conventional prognostic factors which are based upon subjective evaluations of histological type, grade of differentiation, depth of tissue invasion, degree of lymph nodal metastases, and the other factors upon which cancer prognoses are presently based.

The level of c-fyn expression and/or STAT-3 phosphorylation may also serve as a convenient method for tumor grading, to replace or supplement histological grading. A high level of FYN or phospho-STAT-3 in a tumor is indicative of more aggressive disease. Thus, such tumors may be graded accordingly. (Typically, a higher tumor grade number signifies a more malignant tumor.)

The level of c-fyn expression and/or STAT-3 phosphorylation may also be used to assess the metastatic potential of a given tumor. The most aggressive tumors, which are characterized by the highest FYN and/or phospho-STAT-3 levels, are generally expected to be the most metastatic.

The status of STAT-3 molecules present in the cell, i.e. activated or nonactivated, may be determined by convenient assays. According to one embodiment of the invention, a sample is contacted with an antibody specific for phosphorylated STAT-3, which antibody does not significantly cross-react with unphosphorylated STAT-3. The amount of antibody bound by the sample may be determined relative to the amount of antibody bound by a sample of normal tissue of the same type of tissue. The difference in the amount of antibody bound by the normal and test samples is indicative of the patient's prognosis. A low or zero level of antibody binding is indicative of a lower grade of malignancy and a more favorable patient prognosis. Antibody binding levels above that reflected in a normal control sample is indicative of a higher grade of malignancy, and a less favorable patient prognosis.

According to another embodiment of the invention, the presence of activated STAT-3 in the patient sample is determined by a DNA binding assay, utilizing a DNA substrate for STAT-3. One such substrate is the acute phase response element (APRE) high-affinity Sis-inducible element (SIE), which has been shown to bind to STAT-3 with high affinity (Zhong et al., Science 264:95-98 (1994)). The binding sequence for STAT-3 was previously described by Yu et al., Science, 269:81-83 (1995), the entire disclosure of which is incorporated herein by reference. STAT-3 specific oligonucleotides are commercially available (Santa Crux Biotechnology, Santa Cruz, Calif.). One such oligonucleotide has the sequence 5′-GATCCTTCTG GAACCTAGATC-3′ (SEQ ID NO: 1).

According to a STAT-3 DNA binding assay, nuclear extracts are prepared from cells of the patient tumor sample by known techniques, such as the protocol described by Schreiber et al., Nucl. Acids Res. 17, 6419 (1989), the entire disclosure of which is incorporated herein by reference. The nuclear extract is then incubated with appropriately labeled (e.g. radiolabeled) oligonucleotide comprising the phospho-STAT-3 binding site. The resulting DNA-protein complexes are analyzed, such as by an electrophoretic mobility shift assay. See Yu et al., supra. A shift in the mobility of the probe oligonucleotide indicates STAT-3 binding, and thus the presence of the activated, i.e., phosphorylated, form of STAT-3. Unphosphorylated STAT-3 does not bind to the DNA substrate. Mutant oligonucleotides with point mutations in the binding sequence may be used as negative controls. Example 5 herein describes a typical STAT-3 binding assay.

According to another aspect of the invention, the level of c-fyn expression in a patient sample is utilized as a prognostic marker. Determining the relative level of expression of the c-fyn gene in the tissue sample comprises determining the relative number of c-fyn RNA transcripts, particularly mRNA transcripts in the sample tissue, or determining the relative level of the corresponding FYN protein in the sample tissue. Preferably, the relative level of FYN protein in the sample tissue is determined by an immunoassay whereby an antibody which binds FYN protein is contacted with the sample tissue. The relative c-fyn expression level in cells of the sampled tumor is conveniently determined with respect to one or more standards. The standards may comprise, for example, a zero expression level on the one hand and the expression level of the gene in normal tissue of the same patient, or the expression level in the tissue of a normal control group on the other hand. The standard may also comprise the c-fyn expression level in a standard cell line. The size of the decrement in c-fyn expression in comparison to normal expression levels is indicative of the future clinical outcome following treatment.

Methods of determining the level of mRNA transcripts of a particular gene in cells of a tissue of interest are well-known to those skilled in the art. According to one such method, total cellular RNA is purified from the effected cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters by, e.g., the so-called “Northern” blotting technique. The RNA is immobilized on the filters by heating. Detection and quantification of specific RNA is accomplished using appropriately labelled DNA or RNA probes complementary to the RNA in question. See Molecular Cloning: A Laboratory Manual, J. Sambrook et al., eds., 2nd edition, Cold Spring Harbor Laboratory Press, 1989, Chapter 7, the disclosure of which is incorporated by reference.

In addition to blotting techniques, the mRNA assay test may be carried out according to the technique of in situ hybridization. The latter technique requires fewer tumor cells than the Northern blotting technique. Also known as “cytological hybridization”, the in situ technique involves depositing whole cells onto a microscope cover slip and probing the nucleic acid content of the cell with a solution containing radioactive or otherwise labelled cDNA or cRNA probes. The practice of the in situ hybridization technique is described in more detail in U.S. Pat. No. 5,427,916, the entire disclosure of which is incorporated herein by reference. A further example of the application of in situ hybridization is set forth by Mettus et al., Oncogene 9: 3077-3086 (1994), incorporated herein by reference.

The nucleic acid probes for the above RNA hybridization methods can be designed based upon the published c-fyn cDNA sequence of Rigley et al., J. Immunol. 154(3), 1136-1145 (1995) (GeneBank accession no. S74774), the entire disclosure of which is incorporated herein by reference.

Either method of RNA hybridization, blot hybridization or in situ hybridization, can provide a quantitative result for the presence of the target RNA transcript in the RNA donor cells. Methods for preparation of labeled DNA and RNA probes, and the conditions for hybridization thereof to target nucleotide sequences, are described in Molecular Cloning, supra, Chapters 10 and 11, incorporated herein by reference.

The nucleic acid probe may be labeled with, e.g., a radionuclide such as ³²P, ¹⁴C, or ³⁵S; a heavy metal; or a ligand capable of functioning as a specific binding pair member for a labelled ligand, such as a labelled antibody, a fluorescent molecule, a chemolescent molecule, an enzyme or the like.

Probes may be labelled to high specific activity by either the nick translation method of Rigby et al., J. Mol. Biol. 113:237-251 (1977) or by the random priming method, Fienberg et al., Anal. Biochem. 132:6-13 (1983). The latter is the method of choice for synthesizing ³²P-labelled probes of high specific activity from single-stranded DNA or from RNA templates. Both methods are well-known to those skilled in the art and will not be repeated herein. By replacing preexisting nucleotides with highly radioactive nucleotides, it is possible to prepare ³²P-labelled DNA probes with a specific activity well in excess of 10⁸ cpm/microgram according to the nick translation method. Autoradiographic detection of hybridization may then be performed by exposing filters on photographic film. Densitometric scanning of the filters provides an accurate measurement of mRNA transcripts.

Where radionuclide labelling is not practical, the random-primer method may be used to incorporate the dTTP analogue 5-(N-(N-biotinyl-epsilon-aminocaproyl)-3-aminoallyl)deoxyuridine triphosphate into the probe molecule. The thus biotinylated probe oligonucleotide can be detected by reaction with biotin binding proteins such as avidin, streptavidin, or anti-biotin antibodies coupled with fluorescent dyes or enzymes producing color reactions.

The relative number of c-fyn transcripts may also be determined by reverse transcription of mRNA followed by amplification in a polymerase chain reaction (RT-PCR), and comparison with a standard. The methods for RT-PCR and variations thereon are well known to those of ordinary skill in the art.

According to another embodiment of the invention, the level of c-fyn expression in cells of the patient tissue is determined by assaying the amount of the corresponding FYN protein. Similarly, the level of STAT-3 activation may be determined by assaying the amount of phospho-STAT-3 protein.

A variety of methods for measuring the amounts of protein exist, including Western blotting and immunohistochemical staining. Western blots are run by spreading a protein sample on a gel, blotting the gel with a cellulose nitrate filter, and probing the filters with labeled antibodies. With immunohistochemical staining techniques, a cell sample is prepared, typically by dehydration and fixation, followed by reaction with labeled antibodies specific for the gene product coupled, where the labels are usually visually detectable, such as enzymatic labels, florescent labels, luminescent labels, and the like.

According to one embodiment of the invention, tissue samples are obtained from patients and the samples are embedded then cut to e.g. 3-5 μm, fixed, mounted and dried according to conventional tissue mounting techniques. The fixing agent may advantageously comprise formalin. The embedding agent for mounting the specimen may comprise, e.g., paraffin. The samples may be stored in this condition. Following deparaffinization and rehydration, the samples are contacted with an immunoreagent comprising an antibody specific for phospho-STAT-3 or FYN. The antibody may comprise a polyclonal or monoclonal antibody. The antibody may comprise an intact antibody, or fragments thereof capable of specifically binding to phospho-STAT-3 or FYN. Such fragments include, but are not limited to, Fab and F(ab′)₂ fragments. As used herein, the term “antibody” includes both polyclonal and monoclonal antibodies. The term “antibody” means not only intact antibody molecules, but also includes fragments thereof which retain antigen binding ability.

Appropriate polyclonal antisera may be prepared by immunizing appropriate host animals with phospho-STAT-3 or FYN and collecting and purifying the antisera according to conventional techniques known to those skilled in the art. Monoclonal antibody may be prepared by following the classical technique of Kohler and Milstein, Nature 254:493-497 (1975), as further elaborated in later works such as Monoclonal Antibodies, Hybridomas: A New Dimension in Biological Analysis, R. H. Kennet et al., eds., Plenum Press, New York and London (1980). Monoclonal antibodies specific for phospho-STAT-3 and FYN are commercially available. Phospho-STAT-3 antibody is available from New England Biolabs, Inc. (Beverly, Mass.). Anti-FYN may be purchased from Santa Cruz Biotechnologies (Santa Cruz, Calif.).

The antibody either directly or indirectly bears a detectable label. The detectable label may be attached to the primary anti-phospho-STAT-3 or FYN antibody directly. More conveniently, the detectable label is attached to a secondary antibody, e.g., goat anti-rabbit IgG, which binds the primary antibody. The label may advantageously comprise, for example, a radionuclide in the case of a radioimmunoassay; a fluorescent moiety in the case of an immunofluorescent assay; a chemiluminescent moiety in the case of a chemiluminescent assay; or an enzyme which cleaves a chromogenic substrate, in the case of an enzyme-linked immunosorbent assay.

The detectable label comprises an avidin-biotin-peroxidase complex (ABC) which has surplus biotin-binding capacity. The secondary antibody is biotinylated. To locate phospho-STAT-3 or FYN antigen in the tissue section under analysis, the section is treated with primary antiserum against phospho-STAT-3 or FYN, washed, and then treated with the secondary antiserum. The subsequent addition of ABC localizes peroxidase at the site of the specific antigen, since the ABC adheres non-specifically to biotin. Peroxidase (and hence antigen) is detected by incubating the section with e.g. H₂O₂ and diaminobenzidine (which results in the antigenic site being stained brown) or H₂O₂ and 4-chloro-1-naphthol (resulting in a blue stain).

The ABC method can be used for paraffin-embedded sections, frozen sections, and smears. Endogenous (tissue or cell) peroxidase may be quenched e.g. with H₂O₂ in methanol.

The level of phospho-STAT-3 or FYN in tumor samples may be compared on a relative basis to the level in normal tissue samples by comparing the stain intensities, or comparing the number of stained cells. The higher the stain intensity with respect to the normal controls, or the higher the stained cell count in a tissue section having approximately the same number of cells as the control section, the higher the level of phospho-STAT-3 or FYN, and hence the higher the expected malignant potential of the sample.

The practice of the invention is illustrated by the following non-limiting examples.

### EXAMPLE 1

### Materials and Methods

A. Cells, Antibodies and Reagents

The murine hematopoietic cell line 32Dcl3 was maintained in Iscoves modified Dulbecco medium (IMDM) supplemented with 10% fetal bovine serum (FBS) (complete medium) and 1 ng/ml of recombinant IL-3 in a 37° C. incubator with 5% CO₂. The v-src transformed 32D cells (32D/v-Src) were maintained in the same medium but lacking IL-3. The Src dominant negative mutant containing 32D cell line (32D/AMSrc) was generated by transfection of Src mutant cDNA (Kaplan et al., EMBO J. 13:4745-4756 (1994)) in a tetracycline inducible vector (O'Brien et al., Gene 184:115-120 (1997)) with the FLAG epitope at its 3′ end along with PMV-7 vector containing Neomycin resistance gene. Following electroporation, positive clones were selected for G418 resistance and tested for AMSrc expression by western blotting technique using the anti-FLAG antibody. These cells were maintained in complete medium containing 10% IL-3 and 2 μg/ml of tetracycline. The AMSrc protein was induced by culturing cells in the absence of tetracycline for 24-48 h. The JAK2 dominant negative mutant expressing 32D cell line (32D/JAK2KE) was generated by transfection of JAK2KE cDNA (Briscoe et al., EMBO J. 15:799-809 (1995); Kohlhuber et al., Mol. Cell. Biol. 17:695-706 (1997)) in pFLAG-CMV-2 vector (Kodak Scientific Imaging). PMV-7 vector containing Neomycin resistance gene was cotransfected along with it.

The FLAG antibody (D-8), the anti-PI3 kinase antibody (Z-8), the STAT-3 (C-20) antibody and the anti-Src antibody (N-16) were purchased from Santa Cruz Biotechnologies. The monoclonal anti-Src antibody (O5-184) the anti-JAK 2 antibody and the 4G10 anti-phosphotyrosine antibody were purchased from Upstate Biotechnologies. The anti-phosphospecific-STAT-3 antibody was purchased from New England Biolabs. Rabbit anti-mouse antibody (secondary antibody for anti-STAT-3) and mouse monoclonal antibody (secondary antibody for anti-Phosphotyrosine and Src) were purchased from Amersham.

B. Tyrosine Kinase Assay

For kinase assays, cells were collected at each time point, centrifuged and lysed in lysis buffer containing 25 mM HEPES pH 7.6, 0.1% Triton-X100, 300 mM NaCl, 20 mM β-glycerophosphate, 1.5 mM MgCl₂, 0.2 mM EDTA, 2.0 μM DTT, 0.2 mM Na₃VO₄, 2 μg/ml leupeptin and 4 μg/ml aprotinin for 30 min at 4° C. Each lysate containing equal amount of protein (150 μg) was immunoprecipitated (IP) with respective antibody at 4° C. for 2 h followed by incubation with protein A sepharose for 45 min at 4° C. The immunoprecipitates were washed thrice with lysis buffer and once with kinase buffer containing 20 mM HEPES pH 7.6, 20 mM MgCl₂, 20 mM β-glycerophosphate, 20 mM p-nitrophenylphosphate, 0.1 mM Na₃VO₄ and 2 mM DTT. Kinase reactions were performed with 20 μM rATP, 5 μCi of ³²P-γATP in 40 μl kinase buffer for 20 min at 30° C. in the presence of Enolase or the Myelin Basic Protein (MBP) (Chaturvedi et al., Mol. Cell. Biol. 17:3295-3304 (1997); Hibi et al., Genes & Development 7:2135-2148 (1993)). The samples were analyzed using SDS-polyacrylamide gels. Following electrophoresis, the gels were dried and subjected to autoradiography.

C. Western Blotting

For the detection of STAT-3, c-Src, AMSrc and JAK2KE proteins, Western blotting assay was performed. The cells were lysed in a buffer containing 1% NP40 in PBS, 1 mM PMSF, 4 μg/ml aprotinin, 4 μg/ml pepstatin A and 4 μg/ml leupeptin. Equivalent amounts of cell lysates (80 μg) were subjected to SDS-PAGE and the resolved proteins were transferred to Nytran membranes. The membranes were blocked in 5% milk for 30 min at room temperature and then incubated with the primary antibody (anti-STAT-3 antibody or anti-HA antibody or anti-FLAG antibody or anti-Src antibody, N-16) for 1 h at room temperature with constant agitation. The filters were then washed thrice with T-TBST (0.05% Tween-20, 20 mM Tris pH 7.5, 150 mM NaCl) and incubated with secondary antibody (goat anti-rabbit IgG linked to horse radish peroxidase) (1:10000 dilution) for 30 min at room temperature. The reacted bands were detected with enhanced chemiluminescence (ECL, Amersham). For JAK antibodies, antiphosphotyrosine antibody (4G10) and Src monoclonal antibody the manufacturer's instructions for blocking and antibody incubation conditions were followed.

D. Immunoprecipitation

For STAT and JAK kinase phosphorylation assays, normal 32Dcl3 cells and 32D/AMSrc cells, maintained in 1 ng/ml recombinant IL-3 were depleted or IL-3 for 6 h and then stimulated with 1 μg/ml of IL-3 for 10 min. 5×10⁶ cells/sample were lysed for each immunoprecipitation. For STAT phosphorylation assay the cells were lysed in a buffer containing 1% NP40, 1 mM PMSF, 4 μg/ml of aprotinin, 4 μg/ml of pepstatin A and 4 μg/ml of leupeptin in PBS for 30 min at 4° C. For JAK immunoprecipitation, cells were lysed in a buffer containing 25 mM HEPES pH 7.6, 0.1% Triton-X100, 300 mM NaCl, 20 mM β-glycerophosphate, 1.5 mM MgCl₂ 0.2 mM EDTA, 2.0 μM DTT. 0.2 mM Na₃VO₄, 2 μg/ml leupeptin and 4 μg/ml aprotinin. The total cell lysate was immunoprecipitated with anti-STAT3 or JAK specific antibody for 2 h at 4° C. followed by incubation with protein A sepharose for 1 h at 4° C. The immunoprecipitates were then collected by centrifugation, washed thrice with the lysis buffer, dissolved by boiling in Laemmli buffer (Laemmli, Nature 227:680-685 (1970)) and subjected to SDS-PAGE. For v-Src and STAT co-immunoprecipitation assay, lysis buffer was the same as for STAT immunoprecipitations. Equal amount of protein (1 mg) in each sample was immunoprecipitated with anti-Src monoclonal antibody (UBI) for 2 h at 4° C. The immunoprecipitate was then incubated with Protein A Sepharose for 1 h at 4° C. and then collected by centrifugation, washed and subjected to SDS-PAGE.

To perform the assay for peptide neutralization of the immune complex, exponentially growing cells were harvested, pelleted and lysed in the same lysis buffer as mentioned above. Equal amounts of protein (1 mg) was used for immunoprecipitation with anti-Src (N-16) antibody (0.005 μg). Total cell lysates were incubated with anti-Src antibody for 2 h at 4° C. or with antibody preincubated with ten times excess of Src peptide (for 2 h at room temperature) followed by incubation with protein A sepharose for 1 h at 4° C. The immune-complex was then separated on a 10% SDS-polyacrylamide gel and Western blot analysis was performed.

E. Electrophoretic Mobility Shift Assay

Nuclear extracts from 32Dcl3 and 32D/AMSrc cells were prepared using the protocol described by Schreiber et al., Nucl. Acids Res. 17:6419 (1989). For each preparation 5×10⁶ cells were harvested in PBS and centrifuged for 5 minutes at 12000×g to obtain a pellet. The pellet was then suspended in 400-800 μl buffer containing 10 mM Hepes-KOH (pH 7.9), 10 mM KCl, 0.1 mM EDTA, 0.1 mM EGTA, 1 mM DTT, 1 mM PMSF, 2 μg/ml Aprotinin, 2 μg/ml Leupeptin, 2 μg/ml Pepstatin and 2 μg/ml Antipain by gentle pipetting and incubated on ice for 15 minutes. To this suspension 25-50 μl of NP40 was added, vortexed and centrifuged at 12000×g in an eppendorf centrifuge. Both the supernatant and the pellet were saved. The supernatant constituted the cytoplasmic extract while the nuclear pellet was further resuspended in 50-100 μl of cold buffer containing 20 mM Hepes-KOH (pH 7.9), 0.4 mM NaCl, 1 mM EDTA 1 mM EGTA, 1 mM DTT, 1 mM PMSF, 2 μg/ml Aprotinin, 2 μg/ml Leupetin, 2 μg/ml Pepstatin and 2 μg/ml Antipain by mixing vigorously at 4° C. for 15-20 min with a pipette tip. It was then centrifuged at 12000×g for 15 minutes and the supernatant which constituted the nuclear extract was collected.

STAT-3 specific oligonucleotides and their mutant counterpart were purchased from Santa Cruz Biotechnology. The binding sequence for STAT-3 was described by Yu et al. (1995). The sequence of the probes used in the assays was:

| | |
|---|---|
| STAT-3: | |
| 5′-GATCCTTCTGGAATTCCTAGATC-3′ | (SEQ ID NO:1) |
| STAT-3*(mutant): | |
| 5′-GATCCTTCTGGGC*C*G*TCCTAGATC-3′ | (SEQ ID NO:2) |

For STAT-3 mobility shift assays, 7 μg of nuclear extract was incubated with 1 ng of ³²P-labeled probe or with the mutant oligonucleotide in 10 μl of binding buffer (50 mM Tris pH 7.5, 5 mM MgCl₂, 250 mM NaCl, 5 mM EDTA, 2.5 mM dithiothreitol (DTT), 20% glycerine, 50 μg/ml Single Standard DNA, 0.25 μg/ml PolydI.dC) for 30 min at room temperature and electrophoresed on non-denaturing 5% polyacrylamide gels in 0.5% TBE buffer (25 mM Tris, 25 mM Boric acid and 10 mM EDTA) as described by Schreiber et al. (1989). One μg of poly(DI-dC) was used as nonspecific DNA competitor in each reaction. The probe was prepared by end labeling the double-stranded oligonucleotide with [³²P]γATP using T4 nucleotide kinase.

### EXAMPLE 2

### IL-3 Stimulation of Cells Activates c-Src Kinase Activity

The murine hematopoietic cell line 32Dcl3 was used to examine the mechanisms associated with IL-3 mediated activation of myeloid cell proliferation.

The 32Dcl3 cell line was derived from normal mouse bone marrow and is non-tumorigenic (Rovera et al., Oncogene 1:29-35 (1987); Valtieri et al., J. Immunol. 138:3829-3835 (1987)). 32Dcl3 cells are strictly dependent on IL-3 for survival and undergo apoptosis in the absence of this cytokine.

To determine whether c-Src plays a role in IL-3 mediated signal transduction pathways, the in vitro kinase activity of c-Src was assayed in immunoprecipitates derived from 32Dcl3 cells treated with IL-3 for various periods of time.

The 32Dcl3 cells were first cultured in the absence of IL-3 for 6 h and then stimulated with recombinant IL-3 for 10, 15, 30, 60 and 120 min. Following stimulation, cells were lysed and the lysates examined for the presence of endogenous c-Src by the Western blotting technique. In addition, the same lysates were immunoprecipitated with monoclonal antibodies against c-Src and the precipitates subjected to in vitro kinase assays in the presence of an exogenous substrate, Enolase.

Equal amounts of protein (150 μg) in each sample were immunoprecipitated (IP) with 5 μl of Src monoclonal antibody. The immunoprecipitates were washed thrice with lysis buffer and once with kinase buffer containing 20 mM HEPES pH 7.6, 20 mM MgCl₂, 20 mM β-glycerophosphate, 20 mM P-nitrophenylphosphate, 0.1 mM Na₃V0₄ and 2 mM DTT. Kinase reactions were performed with 20 μm rATP, 5 μCi of ³²P-γATP in 40 μl kinase buffer for 20 min at 30° C. Activated Enolase (10 μg/sample) (Boehringer-Mannheim) was used as an exogenous substrate. The samples were analyzed by 12% SDS-PAGE. For direct Western blot analysis 80 μg of protein from each sample was resolved by 12% SDS-PAGE transferred to a nitrocellulose membrane and probed with anti-Src monoclonal antibody.

Cells grown in the absence or presence of IL-3 contained equivalent levels of endogenous c-Src. However, the c-Src protein immunoprecipitated from cells grown in the absence of IL-3 for 6 h did not exhibit any tyrosine kinase activity as measured by phosphorylation of Enolase or autophosphorylation of c-Src. On the other hand, c-Src immunoprecipitates derived from IL-3 stimulated cells showed readily detectable in vitro kinase activity as measured by phosphorylation of Enolase, as well as autophosphorylation of c-Src. Low levels of kinase activity were seen within 10 min. following the addition of IL-3 to the cells and maximal levels of this kinase activity were seen in 30 min. following the addition of IL-3. The results also showed that following the addition of IL-3, c-Src remains in a constitutively activated state as long as IL-3 is present in the growth medium, suggesting that stimulation of 32Dcl3 cells with IL-3 leads to the activation of c-Src kinase activity. Since there is no apparent change in the endogenous levels of c-Src, it appears that this activation occurs exclusively thought post-translational modifications.

### EXAMPLE 3

### Activated c-Src in IL-3 Stimulated Cells Associates with STAT-3

The following experiment was conducted in order to determine whether c-Src in IL-3 stimulated 32Dcl3 cells associates with STAT-3 and whether this association is dependent on its activated state.

For this, 32Dcl3 cells were first cultured in the absence of IL-3 for 6 h and then stimulated with recombinant IL-3 for 10 min. to 2 h. The cell lysates were fractionated on 10% SDS polyacrylamide gels and examined for the presence of c-Src and STAT-3 by Western blot analysis. Cell lysates were then immunoprecipitated with monoclonal antibodies against c-Src or with preimmune serum (PI) and immunoprecipitates were resolved on 10% SDS-polyacrylamide gels. The gels were blotted onto a nytran paper and subjected to Western blot analysis using antibodies against STAT-3.

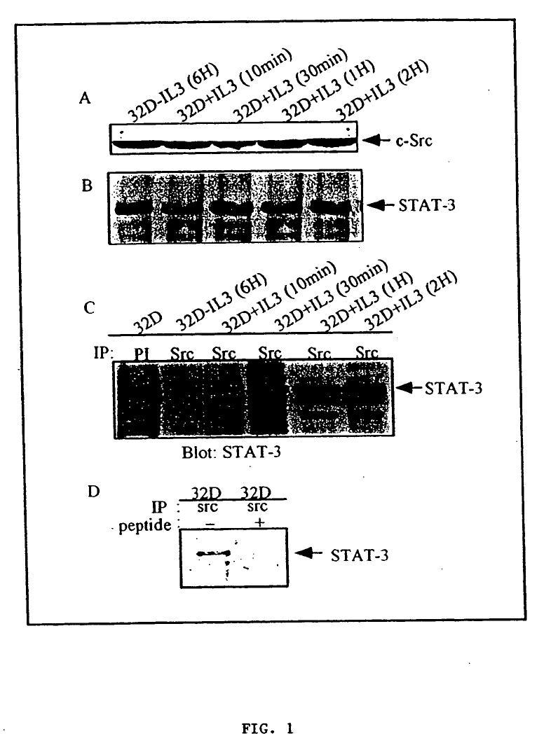

Results from these experiments showed that both c-Src and STAT-3 are present in equivalent amounts in cells grown in the absence of IL-3 as well as in cells grown in the presence of IL-3 for various periods of time (FIGS. 1A and 1B). c-Src immunoprecipitates derived from 32Dcl3 cells grown in the absence of IL-3 did not contain associated STAT-3 protein, even though these cells contained abundant amounts of both c-Src and STAT-3 (FIG. 1C). On the other hand, c-Src immunoprecipitates derived from IL-3 stimulated cells, exhibited association with STAT-3 within 10 min. following the addition of IL-3 to the cells and the levels of associated STAT-3 reached a peak between 30 and 60 min following the addition of the cytokine. Earlier experiments showed that c-Src-associated kinase activity reaches peak levels between 30 and 50 min, so activation of c-Src appears to be essential for its association with STAT-3.

The interaction of c-Src with STAT-3 was also demonstrated using polyclonal antibodies raised against an N-terminal peptide of c-Src. Total cell lysates prepared from 32Dcl3 were immunoprecipitated with anti-Src (N-16) antibody or with antibody preincubated with ten times excess of Src peptide for 2 h at 4° C. The immune-complexes were then resolved on 10% SDS-polyacrylamide gels and immunoblotted using anti-STAT-3 antibody. The results are shown in FIG. 1D. STAT-3 was again co-immunoprecipitated with c-Src. The specificity of c-Src binding to STAT-3 was demonstrated by the ability of the c-Src peptide (against which the antiserum was raised) to block immunoprecipitation of STAT-3.

### EXAMPLE 4

### A Dominant Negative Mutant of Src Blocks IL-3 Induced Activation of STAT-3

To definitively demonstrate that c-Src mediates phosphorylation of STAT-3, 32Dcl3 cells were stably transfected with a tetracycline-inducible dominant negative mutant of Src (AMSrc) (Kaplan et al., EMBO J. 13:4745-4756 (1994)). The ATP-binding site of this c-src mutant was inactivated by mutation of lysine 295 to arginine rendering this protein kinase-inactive. In addition, a phenylalanine substitution for tyrosine 527 prevents the intramolecular interaction between phosphorylated Y527 and the SH2 domain of this protein allowing the protein to exist in an open configuration, thus making the SH2 and SH3 domains accessible to cellular binding proteins (Kaplan et al., 1994). This protein was tagged with the FLAG epitope at the C-terminal end, which allowed the detection of AMSrc independent of endogenous c-Src. Following electroporation of the expression vector into 32Dcl3 cells, the cells were selected with G418 and maintained in a medium containing 2 μg/ml of tetracycline, which blocked the expression of the mutant protein. To verify the inducible expression of AMSrc in 32D/AMSrc cells, the cells were incubated in a medium lacking tetracycline for 24 h and the cell lysates subjected to Western blotting using anti-HA antibodies. As negative controls, cell lysates from parental 32Dcl3 cells and un-induced 32D/Amsrc cells were used.

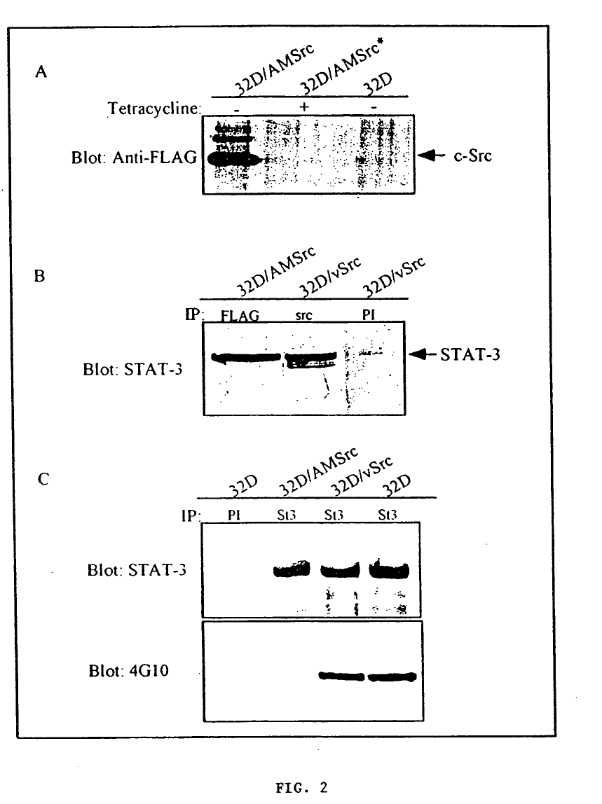

Total cell lysates from normal 32Dcl3 cells and 32D/AMSrc cells cultured in the presence of tetracycline (32D/AMSrc*) or absence of the drug (32D/AMSrc) were fractionated on 12% SDS-polyacrylamide gels and the resolved proteins were immunoblotted with anti-FLAG antibodies. These Western blotting studies (FIG. 2A) showed that the AMSrc protein is not expressed in normal 32Dcl3 or 32D/AMSrc cells grown in the presence of tetracycline. However, high level expression of this protein was induced in 32D/AMSrc cells upon the removal of tetracycline from the culture medium. To determine whether the dominant negative form of Src binds to STAT-3, as was seen with c-Src and v-Src proteins (Yu et al., Science 269:81-83 (1995); Cao et al., Mol. Cell. Biol. 16:1595-1603 (1996); Chaturvedi et al., Mol. Cell. Biol. 17:3295-3304 (1997)), cell lysates were prepared from 32D/v-Src and 32D/AMSrc cells.

Total cell lysates were prepared from 32D/v-Src and 32D/AMSrc cells grown in the absence of tetracycline and 1 mg protein from each sample was immunoprecipitated with anti-FLAG antibody or anti-Src monoclonal antibody or with preimmune serum (PI) for 2 h at 4° C. The immune-complexes were separated on 10% SDS-polyacrylamide gels, blotted and then probed with anti-STAT-3 antibody.

Results from this experiment (FIG. 2B) showed that both v-Src and AMSrc bound equally well with STAT-3 suggesting that AMSrc retains the ability to interact with STAT-3, as was seen with other cellular substrates. To determine the phosphorylation status of STAT-3 in cells expressing AMSrc, total cell lysates from normal 32Dcl3 cells and 32D/AMSrc cells growing in the presence of recombinant IL-3 were prepared. As positive controls, cell lysates from 32D/v-Src cells were used (Chaturvedi et al., Mol. Cell. Biol. 17:3295-3304 (1997)).

Normal 32Dcl3 and 32D/AMSrc cells were first incubated for 6 h in a medium lacking IL-3 and then stimulated with IL-3 for 10 min before lysis. Total cell lysates were prepared from these cells as well as 32D/vSrc cells growing in the absence of IL-3 and the lysates were immunoprecipitated with anti-STAT-3 antibodies. The immunoprecipitates were subjected to Western blotting. The Western blot was first probed with anti-STAT-3 antibodies, which showed that all three cell lines expressed equivalent amounts of STAT-3 (FIG. 2C) The Western blot was stripped and re-probed with 4G10 antibodies which specifically recognize the phosphotyrosine moiety. These studies revealed that this antibody readily recognizes STAT-3 present in normal 32Dcl3 cells growing in the presence of IL-3 and cells transferred with the v-src expression vector (FIG. 2C). However, this antibody failed to recognize STAT-3 in cell lysates derived from 32Dcl3/AmSrc cells cultured in the presence of IL-3. These results show that phosphorylation of STAT-3 following IL-3 stimulation of 32Dcl3 cells is blocked by AMSrc, indicating that STAT-3 is a substrate of c-Src.

### EXAMPLE 5

### Dominant Negative Src Interferes with the DNA Binding Activity of STAT-3

To determine whether AMSrc interferes with the DNA-binding activity of STAT-3, the DNA-binding ability of STAT-3 present in 32Dcl3 and 32D/AMSrc cells was examined. These assays were performed with the acute phase response element (APRE) high-affinity Sis-inducible element (SIE), which was previously shown to bind to STAT-3 with high affinity (Zhong et al., Science 264:95-98 (1994)).

Nuclear extracts were prepared from normal 32Dcl3 cells and 32D/AMSrc cells that were starved for IL-3 for 6 h and then induced with IL-3 for 10 min. Nuclear extracts containing 7 μg of protein were used to carry out EMSA with 10,000 c.p.m. of ³²P-labeled STAT-3 specific and mutant oligonucleotides as described in Materials and methods. A mutant oligonucleotide (indicated by an asterisk) where the binding sequence was altered was used as a negative control.

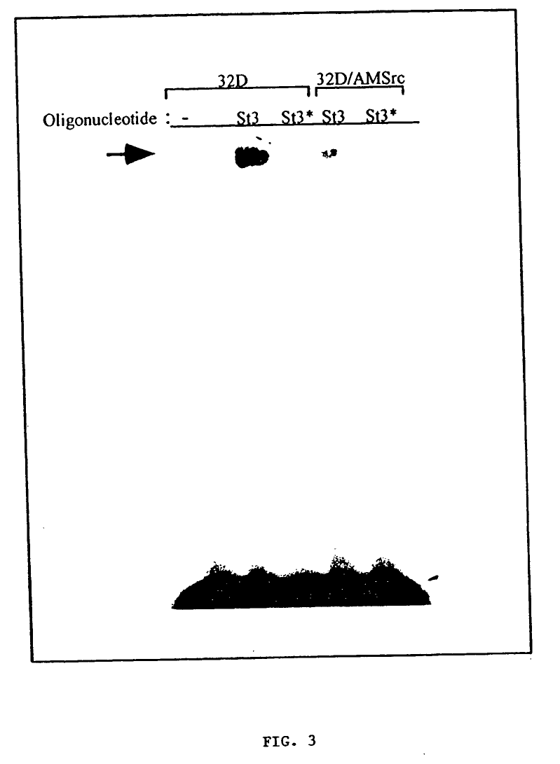

As shown in FIG. 3, nuclear extracts derived from normal 32Dcl3 cells stimulated with IL-3 gave a single shifted band. In addition, a mutant oligonucleotide with point mutations in the binding sequence failed to form such a complex. On the other hand, when these gel shift assays were performed with nuclear extracts derived from 32D/AMSrc cells stimulated with IL-3, significant amounts of a shifted band were not detected. These results show that dominant negative Src interferes with the DNA binding activity of STAT-3, presumably by blocking its phosphorylation on tyrosine.

### EXAMPLE 6

### Effect of Dominant Negative Src on Activation of JAK Kinases

It has been previously shown that interaction of IL-3 with its receptor results in activation JAK2 kinase through an event associated with tyrosine phosphorylation (Silvennoinen et al., Proc. Natl. Acad. Sci. USA 90:8429-8433 (1993)). To determine the status of JAK2 kinase in 32D/AMSrc cells, the levels of JAK2 protein as well as its phosphorylation status were determined and compared to the levels in normal cells. Cell extracts from normal 32Dcl3 and 32D/AMSrc cells were immunoprecipitated with JAK2-specific antibody, subjected to 8% SDS-PAGE, and Western blotted with JAK2 antibody and with monoclonal antibody 4G10. Immunoprecipitates from 32Dcl3 cells stimulated with IL-3 were used as a positive control. 32D/v-Src cells were used as a negative control. These cells grow in the absence of IL-3 and were previously shown not to contain the activated form of JAK2 (Chaturvedi et al., Mol. Cell. Biol. 17:3295-3304 1997)).

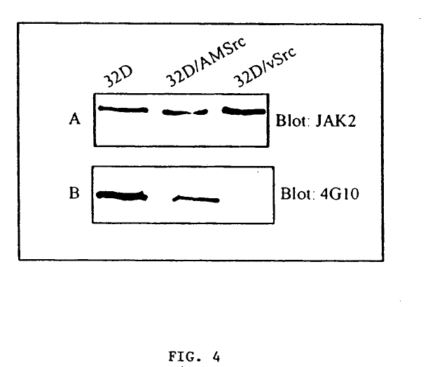

The results from this experiment show that JAK2 is both present (FIG. 4A) and is phosphorylated (FIG. 4B) in both normal 32Dcl3 and 32D/AMSrc cells grown in the presence of IL-3 while it is not phosphorylated in 32D/v-Src cells (which are grown in the absence of IL-3). These results show that IL-3 activates JAK2 phosphorylation and this event does not require the activation of c-Src kinase activity. In addition, these results demonstrate that the presence of activated JAK2 is not adequate to achieve the phosphorylation of STAT-3.

### EXAMPLE 7

### Effect of Dominant Negative Src on IL-3 Induced Proliferation

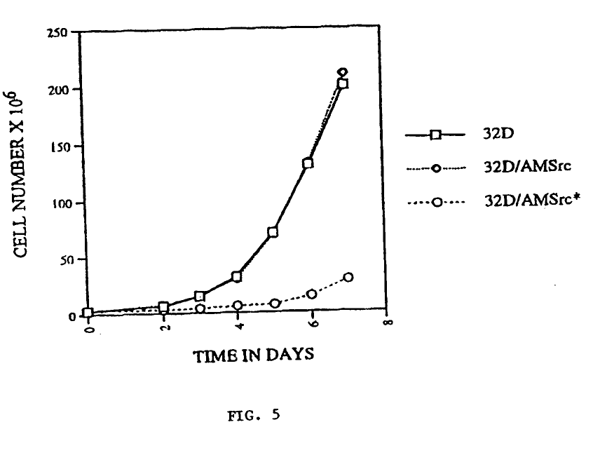

To investigate the effect of AMSrc on 32D cell proliferation, the growth pattern of 32D/AMSrc cells was compared with normal 32Dcl3 cells. Normal 32Dcl3 cells were maintained in IMDM medium supplemented with 10% FBS and 10% WEHI-3B conditioned medium as a source of IL-3. 32D/AMSrc cells were maintained in the same medium but in the presence (32D/AMSrc) or absence of tetracycline (32D/AMSrc*) to repress or induce the expression of AMSrc. The cells were plated at a density of 1×10⁵ cells/ml and cell viability and density determined at 24 h intervals for 7 days. The results are shown in FIG. 5.

In the presence of IL-3, normal 32Dcl3 cells doubled approximately every 24 h and grew in a logarithmic manner and had to be split every 72 h to allow maximal growth. The growth rate of 32D/AMSrc cells was similar to that of normal 32Dcl3 cells, when these cells were grown in the presence of IL-3 and tetracycline (which represses the expression of dominant negative Src). However, when AMSrc protein synthesis was induced by removing tetracycline from the growth medium, their proliferation rate was drastically reduced and the cells doubled approximately every 96 h. As measured by trypan blue staining, viability was found to be >90%. These results show that AMSrc expression in 32Dcl3 cells results in severely reduced proliferation of myeloid cells in the presence of IL-3 and this may be attributed to the absence of an activated form of STAT-3.

### EXAMPLE 8

### Effect of Dominant Negative Mutant of JAK2 on Proliferation of 32Dcl3 Cells

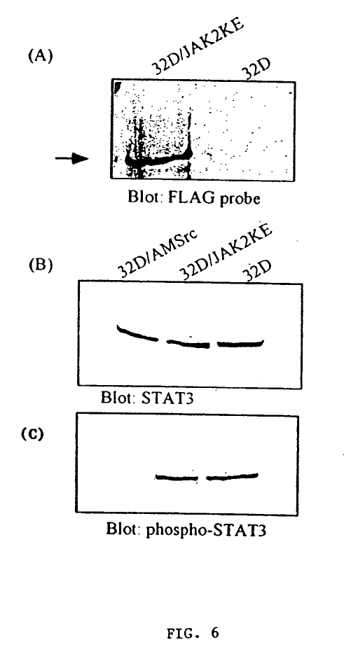

To investigate the role of JAK-2 in IL-3-induced proliferation of 32Dcl3 cells, these cells were stably transfected with a kinase-negative mutant of JAK2 (JAK2KE), which has been previously shown to act in a dominant negative manner (Briscoe et al., EMBO J. 15:799-809 (1995); Kohlhuber et al., Mol. Cell. Biol. 17:695-706 (1997)). This mutant was made by replacing the highly-conserved lysine (K) in motif II of the tyrosine kinase domain with glutamic acid (E). To distinguish the mutant protein from endogenous JAK2, the protein was tagged with the FLAG epitope. Individual clones (32D/JAK2KE) expressing high levels of this protein were selected and were verified for the expression of the mutant protein using antibodies specific to the FLAG epitope. For this, cell lysates were prepared from normal 32Dcl3 and 32D/JAK2KE cells, and 80 μg of total lysate was fractionated by 8% SDS-PAGE. Gels were subjected to Western blotting and probed with anti-FLAG antibody which showed that JAK2KE is expressed at high levels in 32D/JAK2KE cells, while this protein is not seen in normal 32Dcl3 cells (FIG. 6A).

To determine the phosphorylation status of STAT-3 in cells expressing JAK2KE, total cell lysates from normal 32Dcl3 cells and 32D/JAK2KE cells growing in the presence of recombinant IL-3 were prepared. Cell lysates from 32D/AMSrc cells were included as a negative control for STAT-3 phosphorylation. Cells were first incubated for 6 h in a medium lacking IL-3 and then stimulated with IL-3 for 10 min before lysis. The lysates (100 μg) were subjected to Western blotting after 10% SDS-PAGE. The blots were probed with anti-STAT-3 antibody (FIG. 6B), as well as with anti-phospho-STAT-3 antibody which specifically recognizes the phosphotyrosine moiety of STAT-3 (FIG. 6C). These studies revealed that STAT-3 is present in normal 32Dcl3 as well as 32D/JAK2KE and 32D/AMSrc cells growing in the presence of IL-3. However, unlike with AMSrc, IL-3 mediated phosphorylation of STAT-3 was unaffected by the expression of the dominant negative mutant of JAK2 (JAK2KE).

Also unlike with AMSrc, expression of JAK2KE did not affect the ability of 32Dcl3 cells to proliferate in the presence of IL-3. The growth rate of 32Dcl3 cells and 32D/JAK2KE cells was measured as described in Example 7. In the presence of IL-3, both cell lines doubled approx. every 24 h, grew in a logarithmic manner, and were found to be over 90% viable.

### EXAMPLE 9

### Assays to Identify Compounds that Inhibit Cell Proliferation

Based on the observations (see Examples 1-8) that STAT protein activation by tyrosine phosphorylation and subsequent nuclear localization is not mediated as previously thought by the activity of JAK protein kinases, but by Src family kinases, assays are designed for use in identifying small molecule compounds that are specific inhibitors of Src kinase mediated STAT phosphorylation and activation. Since STAT proteins are found to be constitutively activated in a wide variety of human tumors, the interaction between Src kinases and STAT proteins is an effective target for the screening of anticancer therapeutics.

A. Transcription Transactivation Assay Using Constitutive Src Constructs

A reporter construct is used in which transcription of a marker gene (for example Green Fluorescent Protein) is under control of a STAT dependent promoter element, and thus the presence of a functional STAT protein in tyrosine phosphorylated form is required for transcription and subsequent expression of the marker protein. This construct is integrated in single copy at a known locus in a fission yeast genome. Also integrated in single copy under control of constitutively active promoters such as Adh, are an activating Src family kinase cDNA gene (c-Src, c-Fyn, c-Fgr, etc.), and a cDNA gene for the appropriate STAT protein. In the absence of any inhibitory activity, STAT activation occurs via tyrosine phosphorylation by Src kinase, with subsequent transcription transactivation and expression of the marker GFP. Under steady state conditions, average signal strength will reflect maximal STAT phosphorylation and activation achievable for the given expression levels of Src kinase and STAT protein. Cells are treated with a combinatorial library of test compounds. On treatment of cells with a compound exhibiting kinase inhibitory activity, the steady state level of activated STAT protein will decrease, leading to a decrease in the fluorescence signal from GFP.

To control for non-specific changes in GFP steady state levels by, for example, protein synthesis inhibitors or non specific cytotoxic effects, a second marker gene, encoding Blue fluorescent Protein (BFP) is integrated at a separate locus under control of a constitutive promoter such as Adh. The assay readout displays the ratio of light emission at the two wavelengths in the presence and absence of the test compound.

B. Transcription Transactivation Assay Using Inducible Src Constructs

In a variation of Assay A, the appropriate Src kinase is placed under the control of an inducible promoter such as nmt, nmt 41, or nmt 81. The other assay components are as described for Assay A. At the assay starting point the signal from GFP is effectively zero, since transcription of the GFP gene is dependent on expression of a functional Src kinase. At time zero, Src kinase gene expression is induced, and cells are exposed to test compounds during the time course of the assay. Presence of a specific inhibitor will result in a reduction of GFP derived signal relative to, for example, a BFP derived signal at any time point post-induction.

C. Assay Based on the Direct Measurement of Src-Mediated Phosphorylation of STAT

Another class of assay is based on direct measurement of the levels of Src directed tyrosine phosphorylation of STAT in the presence and absence of test compounds.

In this approach, a cDNA gene encoding the appropriate Src kinase, under control of a constitutive promoter such as Adh, is integrated as single copy at a known locus in a fission yeast genome, resulting in constitutive expression of Src kinase activity. A modified STAT protein reporter gene is similarly integrated under control of a constitutive promoter. The modified STAT protein has the following specific features:

It includes the Src target tyrosine phosphorylation motif.

It lacks all nuclear transport signal sequences.

It includes a membrane transport signal sequence.

In this assay, constitutive expression of Src kinase results in steady state levels of Src kinase activity. Newly synthesized STAT reporter can act as a target for Src kinase, but lacking nuclear transport signal sequences it does not relocate to the nuclear compartment. The reporter gene is, instead, targeted to the cell membrane and secreted. Phosphorylation of the secreted STAT reporter protein is monitored extracellularly using antibody specific for the tyrosine phosphorylated form of the target peptide motif.

### EXAMPLE 10

### Immunological Detection of Phospho-STAT-3 in Breast Cancer Cells

A. Cells, Antibodies and Reagents

Human breast carcinoma cell lines, BT20, 126, T47D, MCF-7, ZR75, BT474, 415, and 435 were maintained in Dulbecco's modified eagle medium (DMEM), supplemented with 10% fetal bovine serum (FBS) (complete medium) in a 37° C. incubator with 5% CO₂. STAT-3 (C-20) antibody, which recognizes phosphorylated and non-phosphorylated STAT-3, was purchased from Santa Cruz Biotechnologies and utilized as a positive control. Phospho-STAT-3 antibody was purchased from New England Biolabs. Rabbit anti-mouse antibody (secondary antibody for anti-STAT-3 and antiphospho-STAT-3) were purchased from Amersham International.

B. Western Blotting

The cells were lysed in a buffer containing 0.05% sodium dodecyl sulfate (SDS) and protease inhibitors including 100 μM PMSF, 100 μM sodium ortho vanadate, 4 μg/ml Aprotinin, 4 μg/ml Pepstatin A, and 4 μg/ml Leupeptin. Equivalent amounts of total cell lysates (80 μg) were subjected to 10% SDS-PAGE and the resolved proteins were transferred to NYTRAN membranes. The membranes were blocked in 3% milk for 30 minutes at room temperature and then incubated with primary antibody for two hours at room temperature with constant agitation. The blot was then washed thrice with T-TBST (0.05% Tween-20, 20 mM Tris pH 7.5, 150 mM NaCl) and incubated with secondary antibody (goat anti-rabbit IgG linked to horse radish peroxidase) (1:10000 dilution) for 30 minutes at room temperature and detected with enhanced chemiluminescence (ECL, Amersham), as set forth by Chaturvedi et al., Mol. Cell. Biol. 17:3295-3304 (1997). The blot was also probed with anti-estrogen receptor antibody (ER).

C. Results

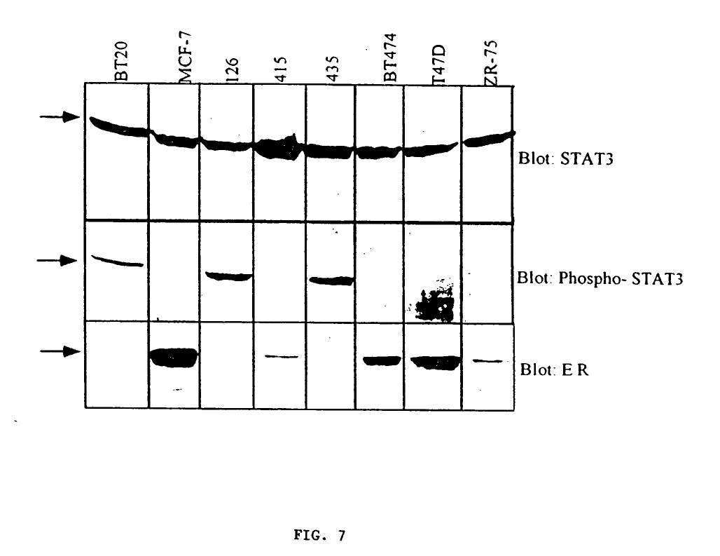

The results are set forth in FIG. 7. The appearance of phospho-STAT-3 correlated with the malignant potential of the cells. Phospho-STAT-3 was present in the estrogen-independent cell lines BT20, 126, and 435, but absent in the estrogen-dependent cell lines MCF-7, 415, BT474, T47D and ZR-75. All cell lines were reactive toward STAT-3 antibody (positive control).

### EXAMPLE 11

### Immunological Detection of Phospho-STAT-3 in Prostate Cancer Cells

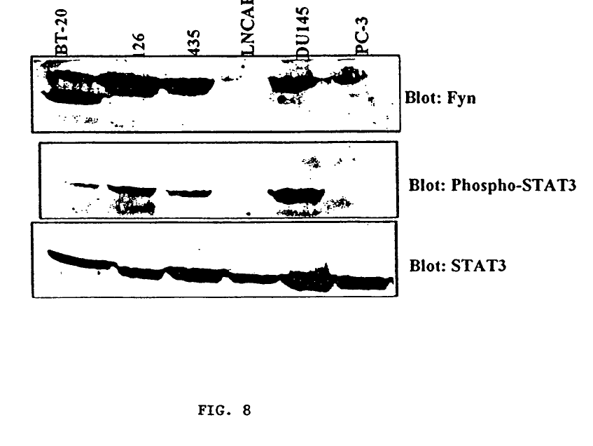

The procedure of Example 10 was repeated, utilizing the prostate tumor cell lines LNCAP, DU145 and PC3. The phospho-STAT-3-positive breast cell lines BT20, 126 and 435 were included as positive controls. The results are set forth in FIG. 8 (bottom two panels). The hormone-independent prostate cancer cell line DU145, but not the hormone-dependent prostate cancer lines LNCAP and PC3, tested positive for phospho-STAT-3.

### EXAMPLE 12

### Immunological Detection of FYN Protein in Breast and Prostate Cancer Cells

The procedure of Examples 10 and 11 was repeated with breast cancer cell lines BT20, 126 and 435, and prostate cancer cell lines LNCAP, DU145 and PC3, substituting anti-Fyn monoclonal antibody (Santa Cruz Biotechnologies) and mouse monoclonal antibody (Amersham International) as the primary and secondary antibodies. The results are set forth in the top panel of FIG. 8. The same estrogen-independent breast cancer cell lines which tested positive for phospho-STAT-3, were likewise positive for FYN. Similarly, the hormone-independent prostate line DU145, which was positive for phospho-STAT-3, was also positive for FYN. The hormone-dependent prostate lines LNCAP and PC3, which were negative for phospho-STAT-3, were likewise negative for FYN. Thus, phospho-STAT-3 and FYN are interchangeable markers for hormone-independence, and therefore malignant potential.

### EXAMPLE 13

### Identification of Antiproliferative Drug Candidate by Phospho-STAT-3 Immunoassay in Breast Cancer Cells

The following demonstrates the identification of a potential anti-cancer agent, FRI-20 (E-4-fluorostyryl 4-chlorobenzyl sulfone), by assaying for the loss of phospho-STAT-3 in the estrogen-independent breast cancer cell line BT20.

A. Preparation of Cell Lysates

BT-20 cells were seeded at a density of 2×10⁶ cells per 100 mm diameter dish and allowed to grow for 24 hours. Fresh medium was added to each plate 2 hours before treatment with the compound. The compound was dissolved in DMSO to make 5 mM stock solution and added to the medium (10 ml) to obtain a final concentration of 25 μM. DMSO alone was added to the control cells to study the effect of vehicle. After 48 hours at 37° C., cells were washed twice with ice cold phosphate-buffered saline and harvested in 400 μl of lysis buffer containing 0.5% SDS, 4 μg/ml aprotinin, 4 μg/ml leupeptin, 4μg/ml pepstatin, 100 μM phenylmethylsulfonyl chloride and 100 μM sodium ortho vanadate. The cells lysates were boiled for 5 minutes and centrifuged for 10 minutes in a micro centrifuge (16000×g). The cell lysates were separated from debris and normalized for protein content.

B. Western Blot Analysis

Equal amounts total protein (100 mg) were run in each lane of SDS-PAGE gel (10%) and transferred to IMMOBILON-P (Millipore, USA). Following the transfer, the membrane was blocked in 3% milk, then probed with phospho-STAT-3 rabbit polyclonal antibody (New England Biolabs) (1:1000) overnight. The membrane washed three times with T-TBS buffer containing Tween-20, Tris HCl (pH7.5) and sodium chloride and probed with horseradish peroxidase-linked donkey anti-rabbit Ig secondary antibody (Amersham International) (1:10,000). The antibody was detected using the ECL Western blotting analysis kit (Amersham International) following the manufacturer's instructions.

C. Results

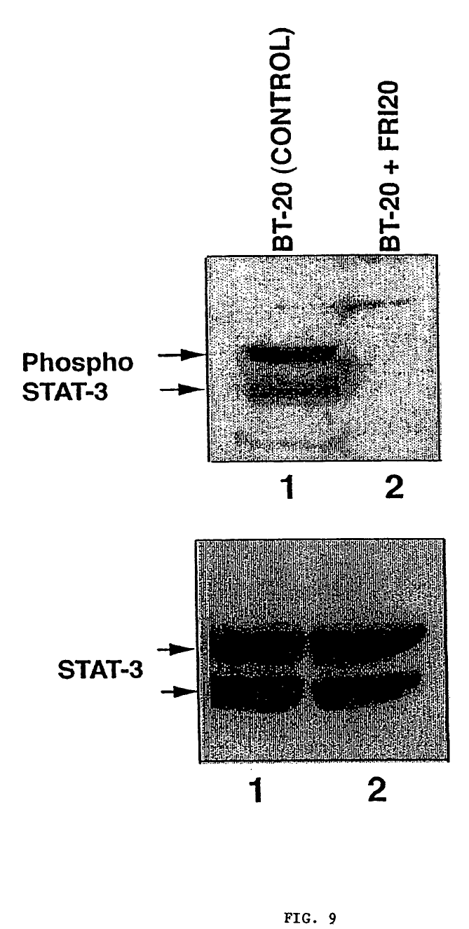

The results are set forth in FIG. 9. Untreated BT20 cells stained positive for phospho-STAT-3, while FRI-20-treatment caused a complete loss of phospho-STAT-3. The loss of phospho-STAT-3 is attributable to an interruption in STAT-3 phosphorylation, not loss of STAT-3 expression, as FRI-20-treated cells stained positive for STAT-3.

In other experiments, FRF-20 dissolved in DMSO at a concentration of 2.5 μM was shown to kill more than 95% of BT20 cells (determined by Trypan blue exclusion) within 72 hours of treatment.

All the references discussed herein are incorporated by reference. Some or all of the reagents, compositions, and supplies needed to carry out the methods, procedures, and techniques disclosed herein may be provided in the form of a kit. Such kits are another embodiment of the present invention.

One skilled in the art will readily appreciate that the present invention is well adapted to carry out the ends and advantages mentioned, as well as those inherent therein. The nucleic acids, compositions, methods, procedures, and techniques described herein are presented as representative of the preferred embodiments, and are intended to be exemplary and not limitations on the scope of the invention. The present invention may be embodied in other specific forms without departing from the spirit or essential attributes thereof and, accordingly, reference should be made to the appended claims, rather than to the foregoing specification, as defining the scope of the invention.

1. A method for determining a prognosis in a patient afflicted with cancer comprising determining the expression level of the c-fyn gene in a sample from the patient, an increased level of c-fyn expression being indicative of an unfavorable prognosis.

2. A method for grading a cancer comprising determining the level of expression of the c-fyn gene in a sample of tissue from a patient suffering from cancer, the level of expression being indicative of the grade of the cancer.

3. A method for determining the metastatic potential of a cancer in an afflicted patient comprising determining the level of c-fyn expression in a sample from the patient, an increased expression level being indicative of the metastatic potential of said tumor.

4. A method according to claim 1, 2 or 3 wherein determining the expression level of the c-fyn gene comprises determining the relative number of RNA transcripts of the gene.

5. A method according to claim 1, 2 or 3 wherein determining the expression level of the c-fyn gene comprises determining the relative level of the FYN protein.

6. A method according to claim 5 wherein the level of the FYN protein is determined by contacting the sample with an antibody which binds the FYN protein.

7. A method according to claim 1 wherein the cancer is a breast cancer.

8. A method according to claim 1 wherein the cancer is a prostate cancer.

9. A method according to claim 1 wherein the cancer is an ovarian cancer.

10. A method according to claim 1 wherein the cancer is a lung cancer.

11. A method for determining a prognosis in a patient afflicted with cancer comprising determining the level of activated STAT-3 protein in a sample from the patient, an increased level of said protein being indicative of an unfavorable prognosis.

12. A method for grading a cancer comprising determining the level of activated STAT-3 protein in a sample of tissue from a patient suffering from cancer, the level of said activated protein being indicative of the grade of the cancer.

13. A method for determining the metastatic potential of a cancer in an afflicted patient afflicted comprising:
1. obtaining a sample of tumor tissue from the patient;
2. obtaining a sample of normal tissue from the patient; and
3. determining the level of activated STAT-3 protein in the sample of tumor tissue and in the sample of normal tissue from the patient,
wherein an increased level of the activated STAT-3 protein in the tumor tissue as compared to the normal tissue indicates an increased metastatic potential of said tumor.

14. A method according to claim 11, 12 or 13 wherein determining the level of activated STAT-3 protein comprises determining the relative level of STAT-3 DNA binding activity.

15. A method according to claim 13 wherein determining the level of activated STAT-3 protein comprises determining the relative level of phosphorylated STAT-3 protein.

16. A method according to claim 15 wherein the level of phosphorylated STAT-3 protein is determined by contacting the sample with an antibody which binds said phosphorylated STAT-3 protein.

17. A method for identifying compounds that inhibit cell proliferation comprising measuring the ability of a test compound to inhibit Src kinase-mediated STAT phosphorylation, wherein inhibitors of cell proliferation are identified as inhibitors of Src-mediated STAT phosphorylation.

18. The method of claim 17 wherein the Src kinase is selected from the group consisting of c-Src, c-Fyn, and c-Fgr.

19. The method of claim 18 wherein the Src kinase is c-Src.

20. The method of claim 17 wherein the STAT is STAT-3.

21. The method of claim 17 wherein the STAT is STAT-5.

22. The method of claim 17 wherein Src-mediated STAT phosphorylation is measured in a recombinant cell.

23. The method of claim 22 wherein the cell is a fission yeast cell.

24. The method of claim 22 wherein the cell is a mammalian cell.

25. The method of claim 17 wherein Src-mediated STAT phosphorylation is measured in a cell free system.

26. The method of claim 17 wherein the level of inhibition of STAT phosphorylation is measured as the level of expression of a reporter gene under the control of a STAT dependent promoter element.

27. The method of claim 26 wherein the reporter gene encodes green fluorescent protein (GFP).

28. The method of claim 17 wherein the level of STAT phosphorylation is measured directly.

29. The method of claim 17 wherein the level of STAT phosphorylation is measured in a DNA binding assay.
